(19) United States
(12) Patent Application Publication
(10) Pub. No.: US 2014/0012387 A1
(43) Pub. Date: Jan. 9, 2014

(54) SPINAL SPACER DEVICES, TOOLS, AND METHODS

(71) Applicants: Paul Glazer, Chestnut Hill, MA (US); Joseph Ting, Acton, MA (US)

(72) Inventors: Paul Glazer, Chestnut Hill, MA (US); Joseph Ting, Acton, MA (US)

(21) Appl. No.: 14/024,317

(22) Filed: Sep. 11, 2013

Related U.S. Application Data
(63) Continuation of application No. 13/324,101, filed on Dec. 13, 2011.

(51) Int. Cl. A61F 2/44; A61F 2/46

(52) U.S. Cl. CPC A61F 2/442; A61F 2/4611
USPC 623/17.16

(57) ABSTRACT

A spacer device for spinal fixation, such as in corpectomies and discectomies, includes a core and two end plates mounted to the core. One end plate is mounted to each respective end of the core. The core can be configured and adapted to be adjustable to allow for contraction and/or distraction of the end plates. The end plates can include apertures therethrough to facilitate osseointegration of the spacer device within a patient's spine. The angle of the endplates can each be adjusted with respect to the core of the spacer device, for example to accommodate lordosis. A containment mesh is included around the spacer device, wrapping circumferentially around the end plates, and spanning from end plate to end plate. The containment mesh defines a containment volume between the end plates for containing bone replacement materials such as allograft or man-made materials.

### CROSS-REFERENCE TO RELATED APPLICATIONS

This application is a continuation of U.S. patent application Ser. No. 13/324,101 filed on Dec. 13, 2011, which claims the benefit of and priority to U.S. Provisional Patent Application No. 61/427,500, filed Dec. 28, 2010, both of which are incorporated by reference herein in their entirety.

### BACKGROUND OF THE INVENTION

1. Field of the Invention

The present invention relates to orthopedic surgical procedures, and more particularly to tools, methods, and spinal spacer devices for spinal surgeries such as corpectomies, discectomies, and the like.

2. Description of Related Art

A variety of devices are known in the art for fixation to the spine in surgical procedures for treating spinal conditions, such as corpectomies and discectomies. Among these devices are spacer devices such as those shown and described in U.S. Pat. No. 6,296,665 to Strnad et al., which can be used to maintain space between vertebrae after surgical removal of a spinal disc and/or vertebra. These fixation devices include upper and lower platforms joined by a core perpendicular thereto. The platforms have apertures therethrough for allowing surrounding bone to integrate with the fixation device. These devices are used to stabilize the spine and can be used to house bone or bone substitutes to help replace the natural structures removed from the spine. Bone may be held in place by an outer sleeve which is slipped around the apparatus.

Such conventional methods and systems have generally been considered satisfactory for their intended purpose. However, there is still a need in the art for tools, methods, and devices that allow for improved containment of bone, bone substitutes, and the like, and that allow for improved visualization thereof. There also remains a need in the art for such devices, tools and methods that can facilitate adjustments to account for lordosis and the like. The present invention provides solutions for these problems.

### SUMMARY OF THE INVENTION

The subject invention is directed to a new and useful spacer device for spinal fixation, such as in corpectomies and discectomies. The spacer device includes a core and two end plates mounted to the core, one plate mounted to each respective end of the core. The core can be configured and adapted to be adjusted to allow for contraction and/or distraction of the end plates. The end plates can include apertures therethrough to facilitate osseointegration of the spacer device within a patient's spine. In certain embodiments, the angle of the endplates can each be adjusted with respect to the core of the spacer device, for example to accommodate lordosis. In accordance with another aspect, a containment mesh can be included around the spacer device, wrapping circumferentially around the end plates, and spanning from end plate to end plate. The containment mesh can thus define a containment volume between the end plates for containing bone replacement materials such as allograft or man-made materials.

The invention also provides a tool for implanting spacer devices as described above. The tool includes means for adjusting the core of the spacer device to cause collapse/distraction of the endplates. The tool also includes means for clamping and releasing the spacer device. Means are also included for providing control to lock and unlock the spacer device.

The invention also includes a method of using the spacer device and tool described above. The method includes adjusting the angle of the end plates relative to the core of the spacer device, clamping the spacer device with a tool for placing the spacer device, positioning the spacer device in a patient's spine, using the tool to distract the end plates of the spacer device, unclamping the spacer device to release the spacer device from the tool, placing bone replacement materials in a containment volume of the spacer device, and securing the bone replacement materials in the containment device by securing a mesh around the spacer device, wherein the mesh wraps around the spacer device and extends from end plate to end plate thereof.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

So that those skilled in the art to which the subject invention appertains will readily understand how to make and use the devices and methods of the subject invention without undue experimentation, preferred embodiments thereof will be described in detail herein below with reference to certain figures, wherein:

FIG. 1 is a perspective view of an exemplary embodiment of a spacer device constructed in accordance with the present invention, showing the adjustable endplates and core without the containment mesh in place;

FIG. 2 is a side elevation view of the spacer device of FIG. 1, showing the end plates in a first position, for example prior to being adjusted to fit a patient's spine;

FIG. 3 is a side elevation view of the spacer device of FIG. 2, showing the end plates in a second position for accommodating lordosis, for example;

FIG. 4 is an exploded perspective view of a portion of the spacer device of claim 1, showing the attachment of the lower or inferior end plate to the core of the spacer device;

FIG. 5 is a side elevation view of the spacer device of FIG. 1, showing the containment mesh being attached around the end plates;

FIG. 6 is a side elevation view of the spacer device of FIG. 5, showing the containment mesh attached around the end plates;

FIG. 7 is a perspective view of the spacer device of FIG. 1, showing the spacer device positioned at the distal end of an exemplary embodiment of a tool for positioning the spacer device and making adjustments thereto during surgery;

FIG. 8 is a perspective view of the tool of FIG. 7, showing the tool without a spacer device; and

FIG. 9 is a side elevation view of the spacer device and tool of FIG. 7, showing the controls of the tool.

### DETAILED DESCRIPTION OF THE PREFERRED EMBODIMENTS

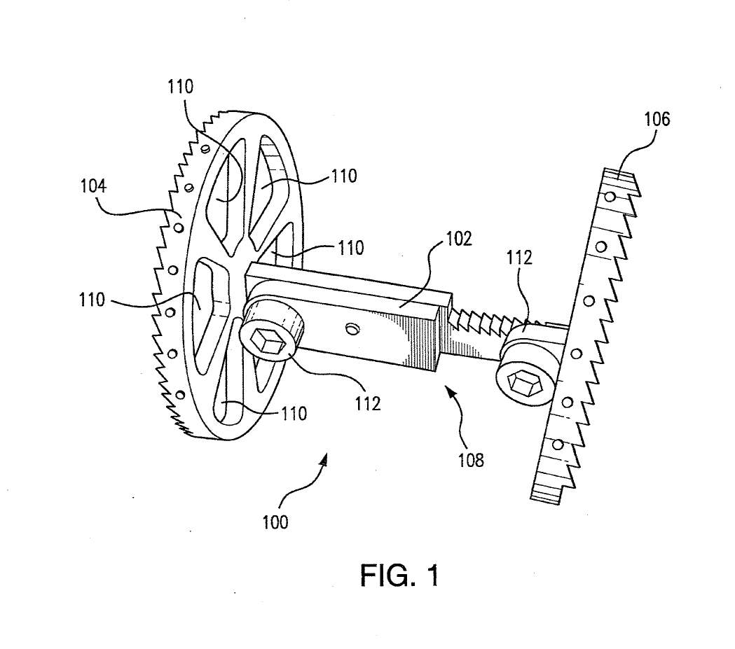

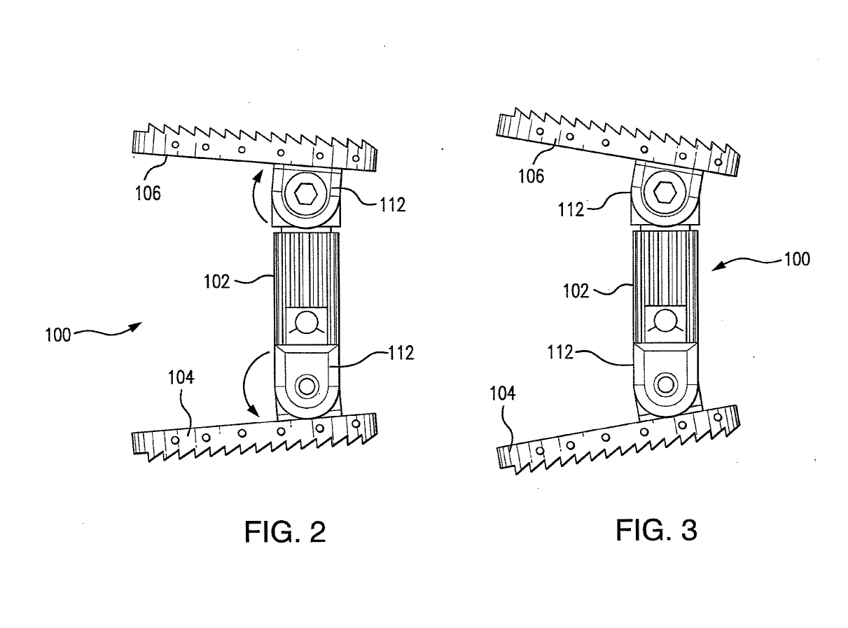

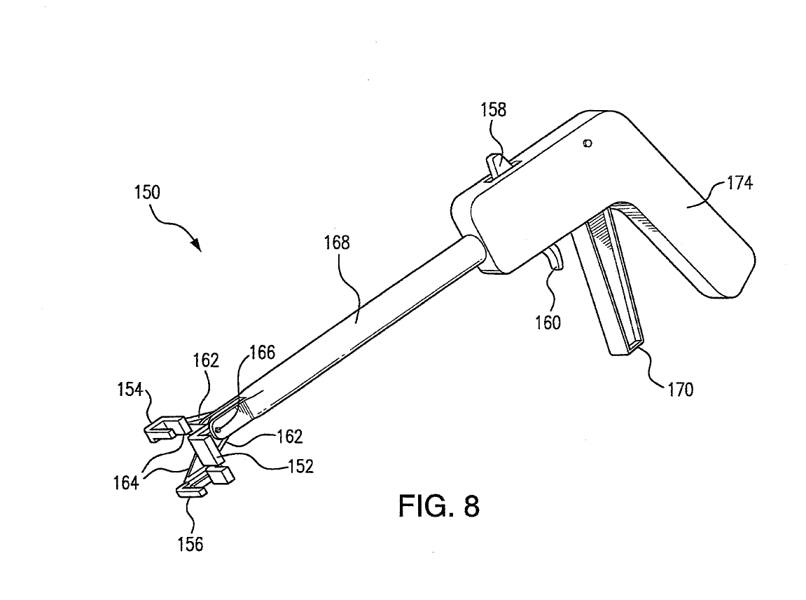

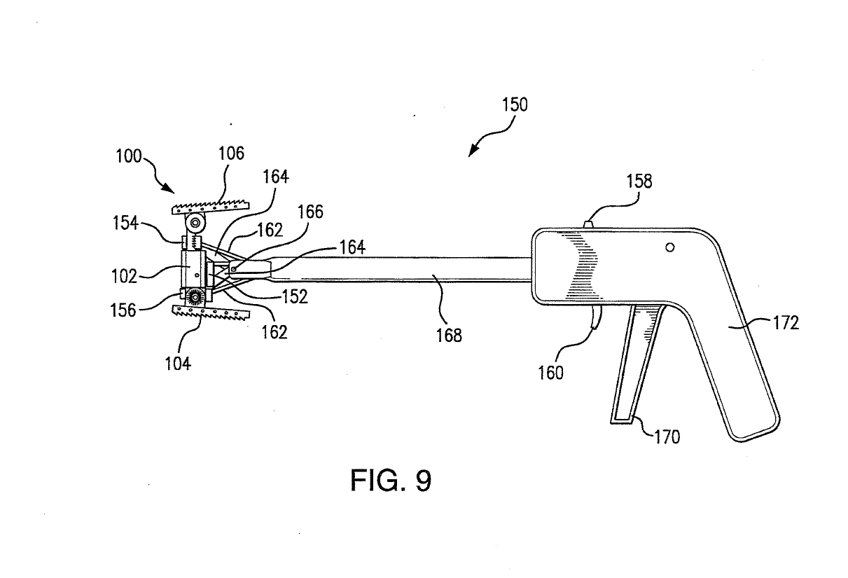

Reference will now be made to the drawings wherein like reference numerals identify similar structural features or aspects of the subject invention. For purposes of explanation and illustration, and not limitation, a partial view of an exemplary embodiment of a spacer device constructed in accordance with the invention is shown in FIG. 1 and is designated generally by reference character 100. Other embodiments of spacer devices in accordance with the invention, or aspects thereof, are provided in FIGS. 2-9, as will be described. The systems of the invention can be used to improve spinal surgeries such as corpectomies, discectomies, and the like.

Referring now to FIG. 1, spacer device 100 includes a core 102 and two end plates 104 and 106 mounted to core 102. The first end plate 104 is mounted a first end of core 102, and second end plate 106 is mounted to the opposite end of core 102. Core 102 includes a ratchet mechanism 108 that is adjustable to allow for contraction and/or distraction of end plates 104 and 106. The neck, i.e., the weakest part of core 102 in ratchet mechanism 108, is configured to withstand between 500 lbf and 1000 lbf in compression. End plates 104 and 106 include apertures 110 therethrough to facilitate osseointegration of spacer device 100 within a patient's spine.

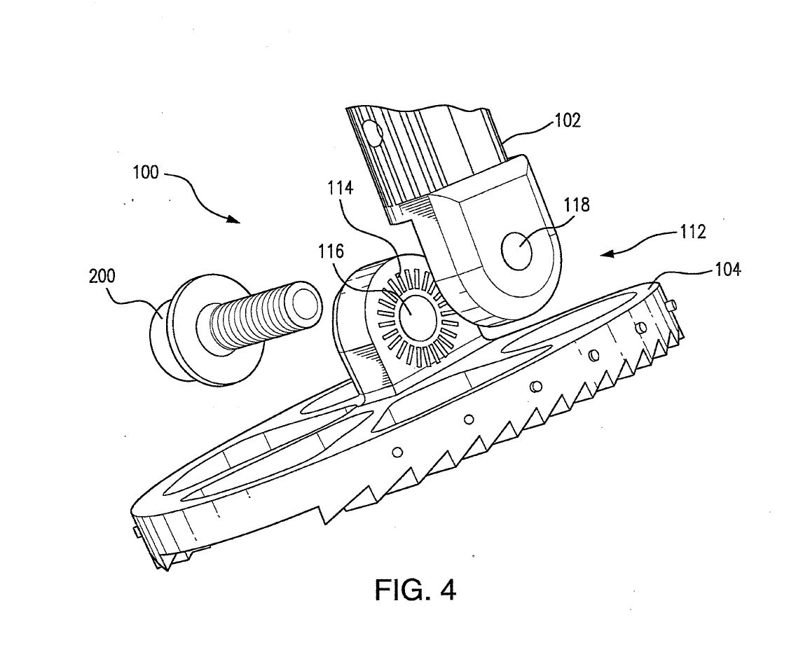

Each end plate 104 and 106 forms a mechanism 112 with core 102 that is adjustable so that the angle of end plates 104 and 106 can each be independently adjusted with respect to core 102 of the spacer device. This allows a surgeon to set the plate angles to custom fit spacer device 100 in a patient's spine, such as to accommodate lordosis. FIG. 2 shows spacer device 100 with end plates 104 and 106 nearly parallel to one another, and indicates the directions of motion for mechanisms 112 to place endplates 104 and 106 in the angled positions shown in FIG. 3. FIG. 4 shows one of the mechanisms 112 in an exploded view to show locking features 114, which are a circular array of indentations around aperture 116. A corresponding circular array of protrusions or teeth (not shown) is provided around the corresponding aperture 118 of core 102. The teeth and indentations of locking features 114 can be secured together by tightening fastener 120 in apertures 116 and 118. If it is desired to adjust the angle of plate 104, fastener 120 can be loosened enough to allow locking features 114 to rotate. Once the desired plate angle is attained, it can be locked or secured by tightening fastener 120 to lock locking features 114. End plate 106 is attached to core 102 by a similar fastener and locking features. The teeth of locking features 114 are simple, rounded features, but biased barbed features, friction locking features, or any other suitable locking features can be used without departing from the spirit and scope of the invention.

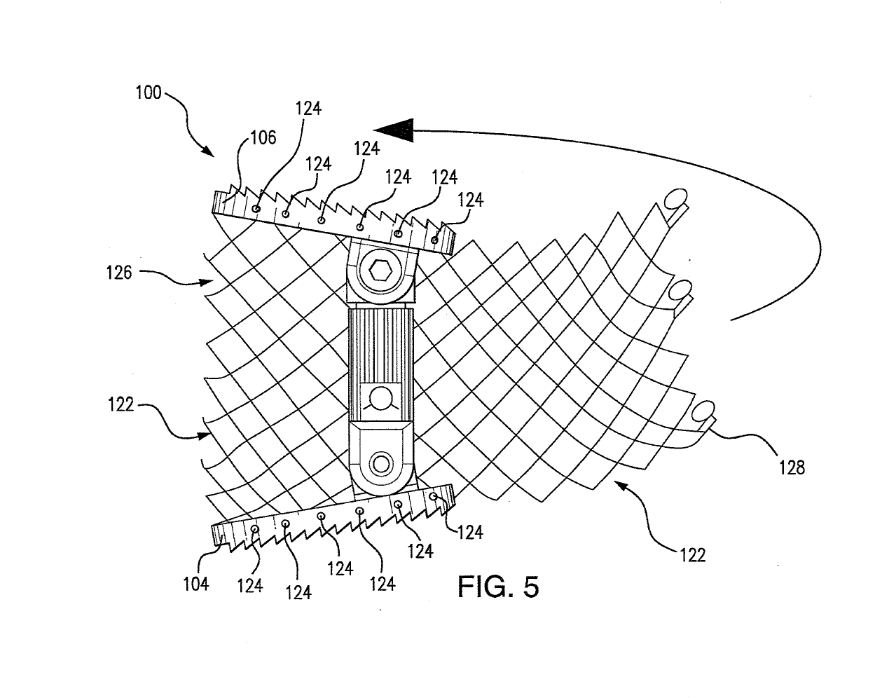

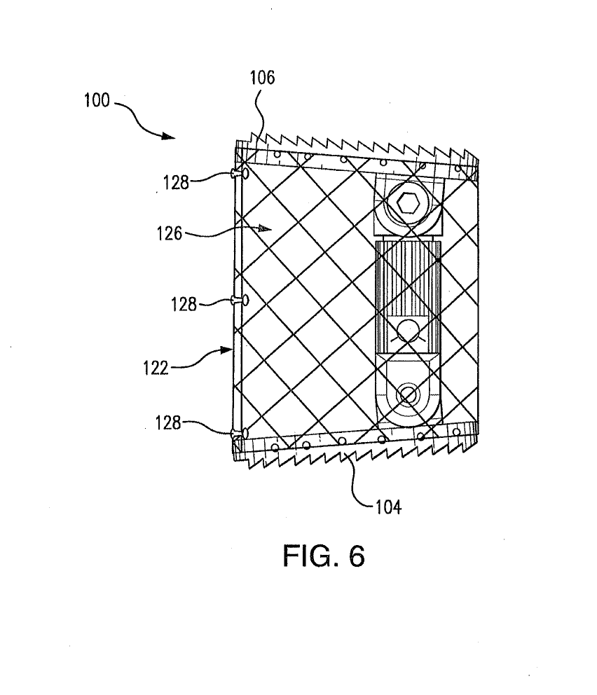

Referring now to FIGS. 5-6, spacer device 100 includes a containment mesh 122 for enclosing a containment volume 126 between end plates 104 and 106 for holding bone replacement materials such as autograft, allograft, biologics such as BMP, synthetic bone matrices, other suitable man-made materials, and/or the like. These materials can integrate with a patient's bone adjacent each end plate 104 and 106 through apertures 110 as described above. Through mesh 122, these materials can be seen and can even be manipulated within containment volume 126 during surgery. For example, mesh 122 allows visualization of the fusion mass and allows containment of the bone graft within containment volume 126 as well. Mesh 122 wraps circumferentially around end plates 104 and 106, spanning spacer device 100 axially from end plate 104 to end plate 106. The circumferential edges of mesh 122 are affixed to end plates 104 and 106 by attachment to pegs 124 protruding therefrom. Bone replacement materials can be placed in containment volume 126 with mesh 122 in the open position shown in FIG. 5.

Once filled to a satisfactory extent with bone replacement materials, containment volume 126 can be closed off by completing the wrapping of mesh 122 around spacer device 100 as indicated by the large arrow in FIG. 5, if it has not already been done. When wrapped around spacer device 100, the axial-running edges of mesh 122 can be secured to each other by hooking hooks 128 to closed mesh cells on the opposed mesh edge, as indicated in FIG. 6. Mesh 122 can be attached to the end-plates using any other suitable mechanical fixation, adhesive, or welding (assuming end plates 104 and 106 and mesh 122 are made from compatible materials) without departing from the spirit and scope of the invention. Examples of mechanical fixation include tying the ends of the lattice to the edges of the end-plates or loop-like structures integrated into the sides of the end plates. Clips may also be used. The lattice of mesh 122 is intended to be attached, but with one edge free so that the lattice does not completely enclose spacer device 100, i.e., leaving an opening for insertion of typical interbody biologic materials, or bone replacement materials, as described above. Closure of the lattice may be performed using hooks 128 or any other suitable mechanical features integrated along a free edge of the lattice. Mesh 122 can be partially applied to the device before insertion, leaving an opening laterally that allows tool 150, described below, to be used for vertical corpectomy device expansion. The lateral opening can be left open, or a flap of mesh can be used, e.g., with a hook and latch fastener, to seal over the lateral opening once bone graft material is placed.

As shown in FIG. 6, the open ends of hooks 128 interlock with open cells along the corresponding free edge of mesh 122. This closure could also be performed using a hook-and-loop fastener as described above with two separate strips, one incorporating a plurality of hooks and the other a series of loops. Another exemplary closure technique includes integration of a fiber into the lattice of mesh 122 to allow a purse-string closure so that closure can be achieved by pulling the two free ends of the fiber together. The lattice could also be closed by suturing the ends of the lattice together. Those skilled in the art will readily appreciate that any suitable mesh closure means can be used without departing from the spirit and scope of the invention.

The lattice of mesh 122 is an open structure with extensibility for conformance to the adjustable end plates 104 and 106. Any suitable pore or cell size can be used, and it is contemplated that the cell size will be variable as spacer device 100 is manipulated. For example, it is contemplated that the relaxed cell size can be about ⅛ of an inch. The lattice can be fabricated using any suitable process, including for example, knit, braid, or other standard textile techniques producing an open structure with extensibility. The lattice can be made using a polymer or metal compatible with the biologic environment. One potential material is polypropylene, the material used to fabricate Prolene sutures. Other exemplary materials include, but are not limited to titanium, or nitinol, PEEK or biodegradable polymers. Individual lattice fibers an have a monofilament structure or can be fabricated by braiding or twisting a plurality of individual fibers. Those skilled in the art will readily appreciate that any suitable materials, lattice fiber structure, cell size, etc., can be used without departing from the spirit and scope of the invention.

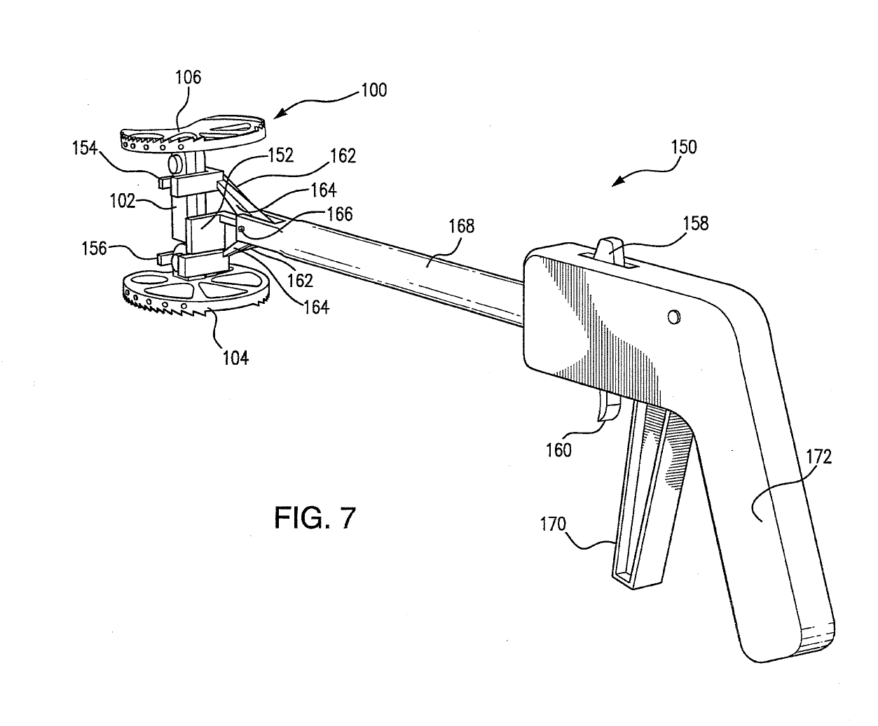

With reference now to FIGS. 7-9, there is depicted a tool 150 in accordance with the invention for implanting spacer devices as described above. Tool 150 includes a pusher 152 which presses against core 102 of spacer device 100 to unlock ratchet mechanism 108 (see FIG. 1). Slider 158 controls pusher 152. Slider 158 can be pushed to unlock ratchet mechanism 108, and can be released to lock ratchet mechanism 108. Tool 150 is configured to prevent the spacer from moving backwards, i.e., pusher 152 is a relatively wide paddle that pushes against an unlocking tab on ratchet mechanism 108 of spacer device 100, but can be withdrawn from the unlocking tab to prevent unwanted collapse of end plates 104 and 106.

Upper and lower grips 154 and 156 grip core 102 of spacer device 100, and trigger 160 can be pulled to open upper and lower clamps 154 and 156 and thereby release spacer device 100 from tool 150. A respective spring on each of the top and bottom clamps 154 and 156 keeps them normally open. Pulling back on trigger 160 opens clamps 154 and 156, and releasing trigger 160 causes clamps 154 and 156 to grasp, e.g., to grasp core 102 of spacer device 100. Upper and lower clamps 154 and 156 are connected to tool 150 by outer linkages 162 and inner linkages 164. Inner linkages 164 are pinned to a stationary point 166 that is stationary with respect to main shaft 168 of tool 150. Outer linkages 162 are pinned to a shaft within main shaft 168 that is axially moveable with respect to main shaft 168, and which is operatively connected to finger control 170. Finger control 170 can thus be manipulated toward or away from handle 172 to move upper and lower clamps 154 and 156 toward or away from one another. Thus, with pusher 152 releasing ratchet mechanism 108, finger control 170 can be used to distract and/or collapse end plates 104 and 106 as needed. This control configuration facilitates blind operation of tool 150.

The invention also includes methods of using the spacer device 100 and tool 150 described above. Spacer device 100 is shown in FIGS. 7-9 without mesh 122 in place for clarity, however it is contemplated that during a surgery, mesh 122 could be in place around spacer device 100, but without hooks 128 secured, as is shown in FIG. 5. The opening left by having hooks 128 unsecured allows for main shaft 168 of tool 150 to pass through mesh 122. With end plates 104 and 106 collapsed together, i.e., with ratchet mechanism 108 collapsed, and with the plate angles for end plates 104 and 106 preset and locked as described above, tool 150 can be used to position spacer device 100 between two adjacent vertebrae in a patient's spine after a corpectomy and/or discectomy as described above. The angel of end plates 104 and 106 can be adjusted before and after spacer device 100 is positioned between the two vertebrae using an appropriate tool such as a screw driver to allow mobility between the endplates and core 102. Slider 158 can be pushed to unlock ratchet mechanism 108, and finger controls 170 can be manipulated to distract end plates 104 and 106 apart from one another as needed to provide the proper spacing in the patient's spine. Slider 158 can be released to lock ratchet mechanism 108 in the distracted position. Trigger 160 can be used to release upper and lower clamps 154 and 156 from core 102 of spacer device 100 to release spacer device 100 from tool 150, which can then be withdrawn through the opening in mesh 122. Mesh 122 can be filled with bone replacement materials as described above. Mesh 122 can then be secured around the bone replacement materials by attaching hooks 128 to the free edge of mesh 122 as described above.

The methods and systems of the present invention, as described above and shown in the drawings, provide for spinal spacer devices, methods, and tools with superior properties including improved containment and visualization of bone replacement materials, and improved adjustability. While the apparatus and methods of the subject invention have been shown and described with reference to preferred embodiments, those skilled in the art will readily appreciate that changes and/or modifications may be made thereto without departing from the spirit and scope of the subject invention.

What is claimed is:

1. A spacer device for spinal fixation, comprising:
a core and two end plates mounted to the core, with one end plate mounted to each respective end of the core, wherein the core is configured and adapted to be adjusted to allow for contraction and distraction of the end plates; and
a containment mesh wrapping circumferentially around the end plates, and spanning from end plate to end plate, wherein the containment mesh defines a containment volume between the end plates for containing bone replacement materials.

2. A spacer as recited in claim 1, wherein each of the endplates is adjustable to change the angle of the endplates with respect to the core of the spacer device to accommodate lordosis.

3. A spacer as recited in claim 1, wherein the containment volume between the end plates for containing bone replacement materials surrounds the core and extends from the core outward to the mesh.

4. A tool for implanting a spacer device as recited in claim 1, comprising:
a) a handle and a main shaft;
b) means for adjusting the core of the spacer device to cause collapse/distraction of the endplates, wherein the means for adjusting are operatively associated with the main shaft;
c) means for clamping and releasing the spacer device, wherein the means for clamping and releasing are operatively associated with the main shaft; and
d) means for providing control to lock and unlock the spacer device, wherein the means for providing control are operatively associated with the main shaft.

5. A method of placing a spacer device as recited in claim 1, comprising:
a) adjusting the angle of the end plates relative to the core of the spacer device;
b) clamping the spacer device with a tool for placing the spacer device;
c) positioning the spacer device in a patient's spine;
d) using the tool to distract the end plates of the spacer device;
e) unclamping the spacer device to release the spacer device from the tool;
f) placing bone replacement materials in a containment volume of the spacer device; and
g) securing the bone replacement materials in the containment device by securing a mesh around the spacer device, wherein the mesh wraps around the spacer device and extends from end plate to end plate thereof.
